(19) United States
(12) Patent Application Publication
(10) Pub. No.: US 2017/0119410 A1
(43) Pub. Date: May 4, 2017

(54) Surgical Snare Device

(71) Applicant: Board of Regents of the University of Nebraska, Lincoln, NE (US)

(72) Inventors: Jason MacTaggart, Omaha, NE (US); Alexey Kamenskiy, Omaha, NE (US)

(73) Assignee: Board of Regents of the University of Nebraska, Houston, TX (US)

(21) Appl. No.: 15/307,403

(22) PCT Filed: Apr. 29, 2015
(86) PCT No.: PCT/US15/28227

§ 371 Date: Oct. 28, 2016

Related U.S. Application Data
(60) Provisional application No. 61/985,730, filed on Apr. 29, 2014.
(60) Provisional application No. 62/105,316, filed on Jan. 20, 2015.

(51) Int. Cl. A61B 17/221

(52) U.S. Cl. CPC A61B 17/221; A61B 2017/00358

(57) ABSTRACT

A surgical snare device for engaging an object in a body passage and that employs multiple loops for snaring the object. The loops, which can be made from a single elongated line or separate lines, are positioned at locations that substantially span across the diameter of the body passage. A tubular membrane cap is included which is made from a liner coupled with structural elements made of expandable wire for support. The membrane cap is selectively deployed from a sheath and radially expandable to envelop the captured object so that withdrawing the object is more secure and any sharp edges on the object do not damage walls of the body passage. The loops and membrane cap can be made from a metal, a metal alloy, or polymer. Perforations can be formed in the liner to allow fluid to pass through the membrane cap.

### CROSS REFERENCE TO RELATED APPLICATIONS

This application claims priority from U.S. Provisional Application Ser. No. 61/985,730, filed Apr. 29, 2014, and U.S. Provisional Application Ser. No. 62/105,316, filed Jan. 20, 2015, the full disclosures of which are hereby incorporated by reference herein for all purposes.

### BACKGROUND OF THE INVENTION

1. Field of Invention

The present disclosure relates in general to a device insertable into a body passage, and which includes an elongated member fashioned into multiple coils for grappling objects in the passage, and a membrane cap that selectively deploys to encapsulate the object.

2. Description of Prior Art

Snares are typically used in body passages, such as blood vessels, and often under X-ray guidance to manipulate or retrieve intravascular devices and objects from within a body passage. Snares are also sometimes used to entrap and withdraw wires placed from another remote location, thereby establishing through wire access for device manipulation, such as is sometimes required for the placement of aortic stent grafts. Snares are also used for retrieving non-native objects such as stents, stent grafts, or vascular filters. Snares generally are made up of one or more loops that can be manipulated in concert with long thin plastic tubes, such as sheaths or catheters. Using two dimensional angiography and fluoroscopy, it can be difficult to navigate the snare to the exact three dimensional location inside the blood vessel, which is often required to engage the object that is to be snared. Moreover, currently known snares leave large gaps in the body passage, such as within the loops themselves, between the loops and luminal wall of the vessel, and between the snare and the object, which can create difficulty when attempting to snag or capture an object within the passage.

### SUMMARY OF THE INVENTION

Disclosed herein are examples of a device and method of capturing an object within a body passage. An embodiment of a surgical snare device is disclosed that includes a sheath, a control line in the sheath, an elongate member coupled with an end of the control line and that is selectively insertable into a body passage, and loops along the elongate member and defined where the elongate member is curved so that portions of the elongate member are in proximity with one another, and that are disposed at locations distal from the control line and that substantially circumscribe the sheath. In an embodiment, the loops are interconnected to one another. A plurality of control lines may optionally be included, wherein a one of the control lines each connect to a one of the loops, and that can optionally be circumscribed within an inner sheath. An annular membrane cap can optionally be included that circumscribes the control line or lines, and that is selectively moveable into a deployed configuration that circumscribes a portion of the loops and defines a barrier between the loops and an inner surface of the passage. In this example, the membrane cap includes a sheet like liner formed into a tubular member and elongate structural elements coupled within the liner, and wherein an open end of the membrane cap has a diameter that is selectively changeable. The membrane cap may be selectively moved from a retracted position within the sheath and to a deployed configuration with a portion extending axially outward past the sheath. In this embodiment a radius of the membrane cap flares outward with distance from the sheath when the membrane cap is in the deployed configuration, and has a trumpet like configuration and with a diameter greater than a diameter of the sheath. Perforations may optionally be formed in the liner. The surgical snare device can further optionally include a control line coupled to the membrane cap for selectively moving the membrane cap into the deployed configuration. The loops and the membrane cap can be disposed within the sheath when being inserted into the body passage.

Also disclosed herein is an example of a surgical snare device that includes an elongated tubular sheath selectively insertable into a body passage, at least one loop deployable in the body passage that are each formed from an elongate filament line, that are controllable by a control line disposed in the sheath, and that occupy spaces across the body passage at a location distal from the sheath, and an annular membrane cap having at least a portion in the sheath, that circumscribes the control line, and that is selectively deployed from within the sheath to circumscribe the at least one loop. In an example, the annular membrane cap defines a protective layer between at least one loop and the walls of the body passage when in the deployed configuration. Multiple loops may optionally be included, and wherein the loops are formed from a single elongated element, and can have differing diameters. Optionally, manipulating the control line changes a diameter of the loop.

A method of snaring an object within a body passage is described herein and which includes providing a surgical snare device that comprises a series of loops, each of the loops formed by a bend in an elongate member, inserting the surgical snare device into the body passage so that the loops are strategically disposed in spaces that span across the body passage at a location proximate to the object, and engaging the object with at least one of the loops. The surgical snare can also include a membrane cap that is made up of a tubular liner, in this example the method further includes, inserting the membrane cap into the sheath, pushing the membrane cap from the sheath, thereby causing a diameter of an opening of the membrane cap to expand, further urging the membrane cap in an axial direction so that the membrane cap is between the object and wall of the body passage to define a protective layer for the wall. Loop control lines can be included that couple to the elongate members, and a membrane cap control line, the method further including controlling the loops by manipulating the loop control lines, and separately controlling the membrane cap by manipulating the membrane cap control line. The loops can be disposed in the sheath when the sheath is disposed into the body passage. In an alternate example, the step of engaging the object with one of the loops involves manipulating a control line that couples to a one of the loops.

### BRIEF DESCRIPTION OF DRAWINGS

Some of the features and benefits of the present invention having been stated, others will become apparent as the description proceeds when taken in conjunction with the accompanying drawings, in which:

FIG. 1A is a partial sectional side view of an example of a snare device disposed in a body passage.

FIG. 1B is an axial sectional view of the snare device disposed in body passage and taken along lines 1B-1B.

FIG. 1C is a partial side sectional view of an example of the snare device being inserted into body passage.

FIG. 2 is a partial sectional side view of an alternate embodiment of the snare device of FIG. 1.

FIGS. 3-5 show in a partial sectional side view alternate embodiments of snare devices each having a membrane cap.

FIG. 6 is a partial sectional side view an example of the snare device of FIG. 1 capturing an object in the body passage and a membrane cap deployed over a portion of the object.

FIG. 7 is a partial sectional side view of an example of a snare device with a snare portion fully enveloped by a membrane cap.

FIG. 8 is a partial sectional side view of an alternate embodiment of a snare device having control lines dedicated to specific snare loops.

FIG. 9 is a partial sectional side view of an alternate embodiment of a snare device having a single control line for multiple snare loops.

While the invention will be described in connection with the preferred embodiments, it will be understood that it is not intended to limit the invention to that embodiment. On the contrary, it is intended to cover all alternatives, modifications, and equivalents, as may be included within the spirit and scope of the invention as defined by the appended claims.

### DETAILED DESCRIPTION OF INVENTION

The method and system of the present disclosure will now be described more fully hereinafter with reference to the accompanying drawings in which embodiments are shown. The method and system of the present disclosure may be in many different forms and should not be construed as limited to the illustrated embodiments set forth herein; rather, these embodiments are provided so that this disclosure will be thorough and complete, and will fully convey its scope to those skilled in the art. Like numbers refer to like elements throughout. In an embodiment, usage of the term “about” includes +/−5% of the cited magnitude. In an embodiment, usage of the term “substantially” includes +/−5% of the cited magnitude.

It is to be further understood that the scope of the present disclosure is not limited to the exact details of construction, operation, exact materials, or embodiments shown and described, as modifications and equivalents will be apparent to one skilled in the art. In the drawings and specification, there have been disclosed illustrative embodiments and, although specific terms are employed, they are used in a generic and descriptive sense only and not for the purpose of limitation.

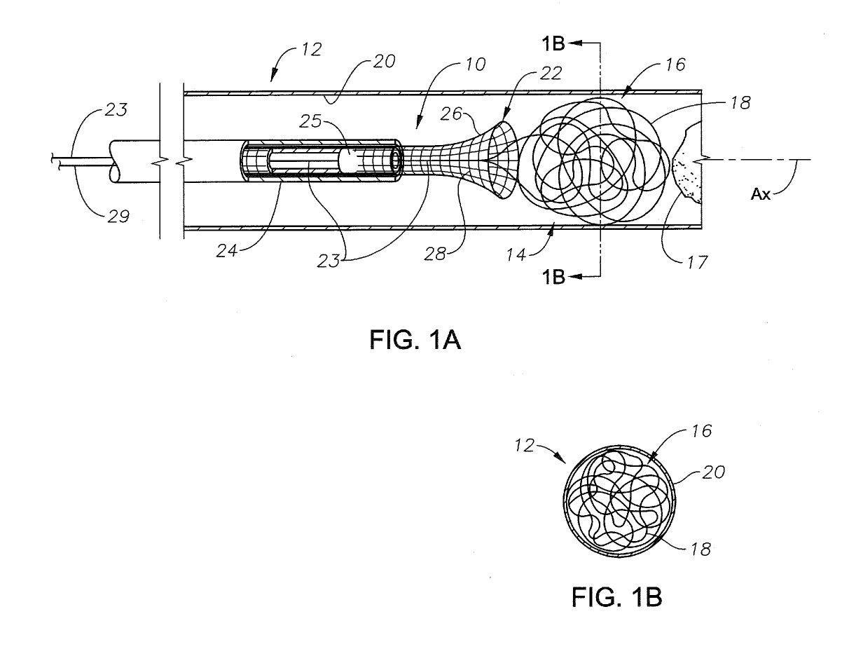

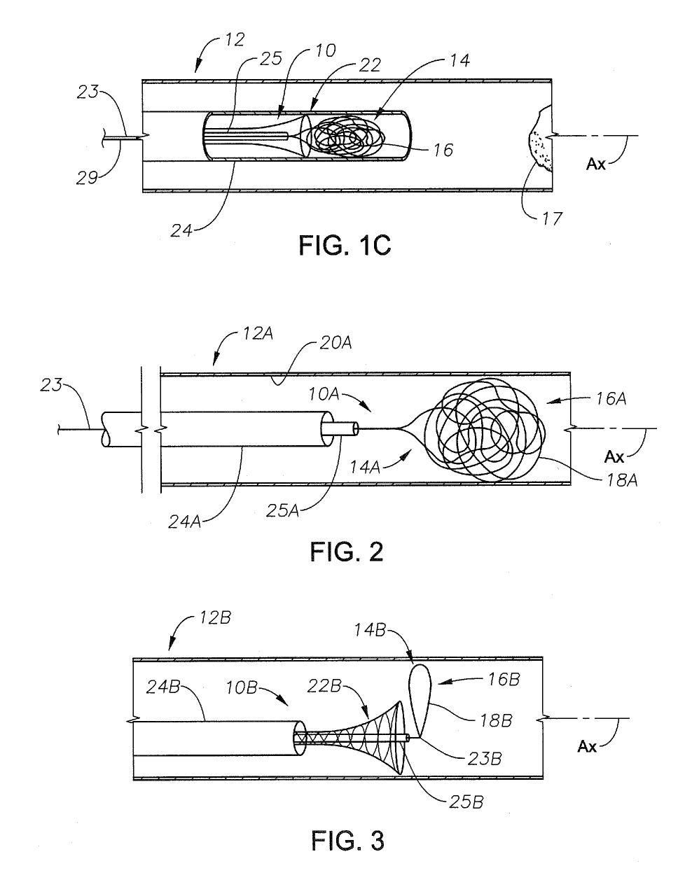

Shown in a side partial sectional view in FIG. 1A is one example of a snare device 10 and which is coaxially inserted into a body passage 12. The body passage 12 could be within a human or animal. Examples exist where the passage 12 is a vessel or tube within the vascular system (such as a blood vessel); is part of a reproductive system, such as a fallopian tube or oviduct; is part of the renal and urinary system, such as an ureter, urethra, and bladder; is part of the lymphatic system; is part of the respiratory system, including bronchus, bronchioles, and alveoli; is part of the gastrointestinal system, such as the intestine, the stomach, gallbladder, cystic duct, bile duct, hepatic duct, pancreatic duct, and the spinal canal/intrathecal. The snare device 10 includes a snare element 14 on one end which is shown made up of a number of loops 16 that may selectively grapple or otherwise engage an object 17 that is within body passage 12. The loops 16 are shown made up of where an elongate element 18 is curved, and where portions of the element 18 come in to close proximity with one another. Embodiments exist wherein all of the loops 16 are made from an element 18 that is a single member, or the loops 16 are made from an element 18 that is made up of multiple members. Further example embodiments exist wherein the loops 16 are separate from one another, or may optionally be interconnected or woven to one another. Example materials used to form the element 18 include alloys of nickel titanium (nitinol), woven nitinol, polymer, monofilament, nanofibers, combinations thereof, and the like. The loops 16 can have a wide range of shapes and circumferences, examples exist wherein the snare element 14 includes 1-20 loops 16, 1-10 loops, or 4-10 loops. The element 18 can also have a wide range of diameters, such as in one example from about 0.06 mm to about 0.12 mm.

Referring now to FIG. 1B, which is taken along lines 1B-1B of FIG. 1A, it can be seen that the loops 16 are provided in locations that span across substantially all of the entire cross-section of body passage 12. Further, in the illustrated example the loops 16 have portions that are in actual contact with a wall 20 formed along an inner surface of body passage 12. An advantage of the strategic placement of the loops 16 throughout the passage 12 is that the ability of grabbing, and retrieving, an object 17 is increased over the use of a lesser number of loops. Referring back to FIG. 1A, an optional membrane cap 22 is shown circumscribing a control line 23 that couples to an end of the snare element 14. As will be described in more detail below, membrane cap 22 can be selectively deployed around the snare element 14 and object 17 and provide a protective barrier for the wall 20. An annular outer sheath 24 is further illustrated and which houses portions of control line 23 and membrane cap 22. Similarly, an optional inner sheath 25 covers the control line 23, and alternatively a portion of snare element 14 proximate its connection to control line 23. In the example of FIG. 1A, inner sheath 25 is between control line and membrane cap 22. Further illustrated in FIG. 1A, is that membrane cap 22 is made up of a sheet-like liner 26 coupled with elongate structural elements that may selectively radially expand when being deployed axially outward from within outer sheath 24 and around snare element 14 and object 17. An example of a matrix web 28 is shown provided in the liner 26, which in one example can be a series of elongate members that are strategically formed within liner 26 to provide structural support for liner 26 and yet still allow for the radial expansion required for membrane cap 22. In an embodiment, the membrane cap 22 is made up entirely of elongate structural elements and without a liner. In an alternative embodiment, the elongate members of the matrix web 28 include wire (potentially nitinol) similar to a stent, or the members could be a wire frame work that are coated. Examples exist wherein the coating on the members of the matrix web 28 is a polymer or fabric of some type. As shown, an opening or mouth of membrane cap 22, depicted on an end projecting outward from outer sheath 24, has a diameter greater than a portion of membrane cap 22 within outer sheath 24. In an example, material of the membrane cap 22 is folds over proximate the circumference of opening to define a cuff (not shown). Moreover, in the illustrated example, the diameter of the membrane cap 22 gradually increases with axial length to initially define a trumpet-like configuration. Further shown in FIG. 1A is a control line 29 exiting an end of outer sheath 24 distal from snare element 14; by selectively actuating control line 29 the membrane cap 22 may be deployed from within sheath, or retracted fully within outer sheath 24.

Shown in a partial side sectional view in FIG. 1C is an example of snare device 10 being inserted into body passage 12. In this embodiment, snare element 14 is fully retracted into outer sheath 24 to prevent it from inadvertently snagging against the side wall 20 of body passage 12. Further retracted into outer sheath 24 is the membrane cap 22. Selective manipulation of control line 23 (within inner sheath 25) can deploy snare element 14 from within outer sheath 24. Similarly, manipulation of control line 29 can deploy membrane cap 22 from within outer sheath 24. Alternatively, outer sheath 24 can be retracted to deploy membrane cap 22. Further selective manipulation of control line 23 can optionally adjust a circumference of one of the loops 16 of snare element 14, such as in an attempt to wrap the one of the loops 16 around an object 17 in order to bind it for retrieval from within the passage 12. Alternatively, loops 16 can be adjusted independently from one another.

Shown in FIG. 2 is a side partial sectional view of an alternative example of snare device 10A and which does not include a membrane cap 22. In this embodiment the loops 16A of the snare element 14A project radially outward and into contact with wall 20A of body passage 12A, and at locations axially distal from outer sheath 24A. Control line 23 is shown exiting inner sheath 25A on an end distal from loops snare element 14A. Similar to the embodiment of FIG. 1A, manipulation of control line 23A can selectively position snare element 14A to capture an object 17 (FIG. 1A) so that object 17 can be withdrawn from passage 12A.

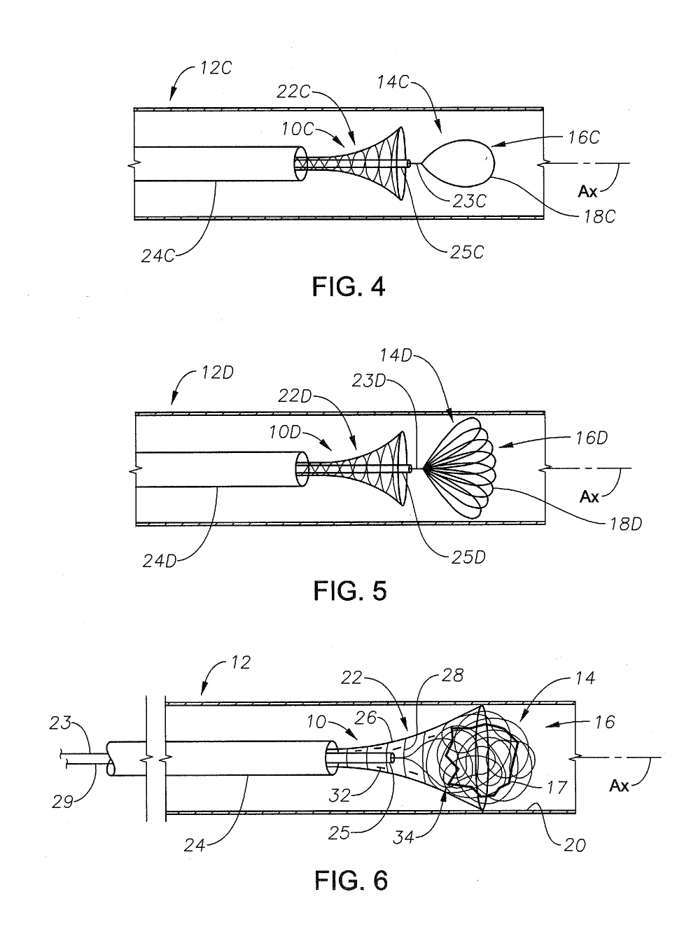

FIG. 3 shows another alternate embodiment of snare device 10B where snare element 14B is a single loop 16B made up of a single element 18B. Control line 23B connects to snare element 14B and extends within inner sheath 25B. Here, a membrane cap 22B is shown having a portion extending axially outward from the outer sheath 24B, further axial extension of membrane cap 22B allows it to radially encompass snare element 14B. In the example of FIG. 3, the loop 16B is disposed in a plane that is generally transverse to an axis A_{X} of body passage 12B. FIG. 4 shows an alternate embodiment of snare device 10C in a side partial sectional view. This example of the snare device 10C has a snare element 14C which includes a single loop 16C made from a single element 18C; and where control line 23B connects to snare element 14B and extends within inner sheath 25B. In this example, a membrane cap 22C is included for selectively encompassing snare element 14C. Here, loop 16C lies in a plane that is generally parallel with an axis A_{X} of body passage 12C. It should be pointed out that embodiments of any of the membrane caps described herein can be used with any type of snare device described herein. Moreover, the membrane caps described herein can be used with any type of snare device, and includes those not described herein.

In another alternate embodiment of snare device 10D shown in side sectional view in FIG. 5, snare element 14D includes a number of loops 16D that are each made from a single element 18D. Here, a single control line 23D connects to ends of each of the loops 16D. Control line 23D is circumscribed by inner sheath 24, that in turn is circumscribed by membrane cap 22D. In this example, loops 16D are in planes that run generally parallel with axis A_{X} of body passage 12D. Examples exist though where loops 16D are disposed in planes that extend perpendicular or oblique to axis A_{X} of body passage 12D. Further examples exist wherein the loops 16D number about eight, or can range in number from about two to about twenty. Thus by engaging object 17 (FIG. 1A) with loops 16D and axially extending membrane cap 22D from within outer sheath 24D, loops 16D and membrane cap 22D can selectively engage object 17 within passage 12D so that object 17 can be removed from passage 12D and without damaging walls of passage 12D.

Referring now to FIG. 6, shown in a side partial sectional view is one example of a membrane cap 22 being deployed axially outward from outer sheath 24. Snare element 14 is anchored to control line 23, which extends through inner sheath 25. Here, matrix web 28 is being radially expanded to encompass snare element 14 and to define a barrier between snare element 14 and wall 20. In this example, membrane cap 22 provides a protective layer for the wall 20 and prevents scratching or other damage to wall 20 either from snare element 14, or from object 17 that is shown being engaged by loops 16 of snare element 14. Examples of object 17 can be a blood clot, tissue, a stent, a stent-graft, a vascular filter, a wire, a vena cava filter, a kidney stone, a swallowed foreign object, as well as a catheter. Embodiments exist where the membrane cap 22 can extend from 6 to 7 centimeters in length. In the example of FIG. 6, perforations 32 are shown formed through the liner 26 and provide a passage for the flow of fluid, such as blood, through liner 26 so that when object 17 and/or snare element 14 is encompassed within membrane cap 22, the object 17 can then be removed from within body passage 12. Optionally, slits, pores, or interstices (not shown) can be provided in the liner 26 in addition to or in place of the perforations 34. Further, object 17 may contain sharp edges 34 on its outer surface that could be damaging to wall 20, thus another advantage of liner 26 is its ability to isolate wall 20 from sharp edges 34. Moreover, separate control lines 23, 29 allow for selective deployment/operation of snare element 14 with control line 23, and selective deployment/operation of membrane cap 22 with control line 29. Thus snare element 14 can be manipulated separately from the manipulation of membrane cap 22 by separate operation of control lines 23, 29. Examples exist wherein membrane cap 22 can retract entirely within outer sheath 24, such as when snare device 10 is being inserted into passage 12. In one example, the opening of the membrane cap 22 can have a diameter that is at least twice that of a diameter of the outer sheath 24. In yet another alternate example, the opening of membrane cap 22 can have a diameter that is at least two to five times a diameter of outer sheath 24. Thus, manipulating control line 23 can deploy and/or retract snare element 14, and also adjust the circumference of one or more loops 16 within snare element 14. Similarly, manipulating control line 29 can deploy and/or retract membrane cap 22, and also adjust the size of the opening of membrane cap 22, i.e. splay it open so that it can encompass snare element 14 and or object 17 as well as reduce its diameter for reinsertion back into outer sheath 24 or held stationary outside the opening of outer sheath 24 if the diameter of captured object exceeds the diameter of outer sheath 24. In one alternate embodiment, material making up web matrix 28 is compressed when being inserted into outer sheath 24, thus by axially retracting sheath from an end of membrane cap 22, the circumferential restraint of the outer sheath 24 is removed allowing web matrix 28 to expand radially outward and without the need for a control line to cause the expansion.

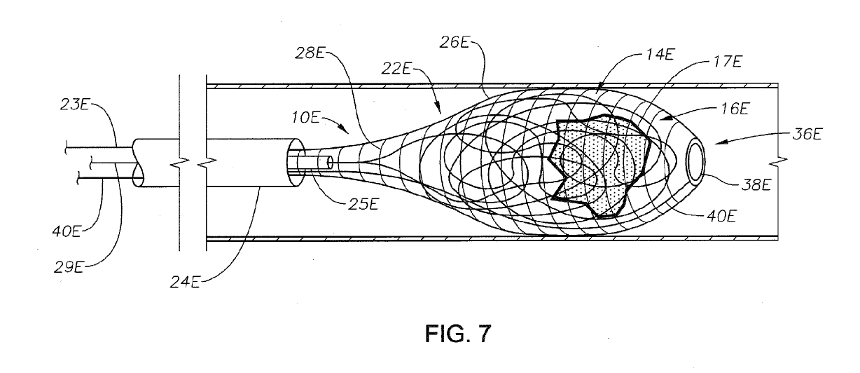

FIG. 7 shows in a side partial sectional view one optional embodiment of snare device 10E wherein membrane cap 22E has been deployed such that it extends axially past all of the snare element 14E and its coils 16E, as well as object 17E and covers a terminal end of snare element 14E distal from the open end of outer sheath 24E. Membrane cap 22E is substantially closed on this terminal end 36E to define a small opening, a loop 38E around opening can be selectively opened and closed with control line 40E shown passing through loop 38E and spanning across membrane cap 22E and back into outer sheath 24E to modify the diameter of the cap opening. Control lines 29E and 40E are shown exiting an end of outer sheath 24E distal from snare element 14E, so that personnel operating snare device 10E can separately operate each membrane cap 22E, and control line 40E. Control line 23E is shown exiting inner sheath 25E, and by selective manipulation of which snare element 14E can be controlled.

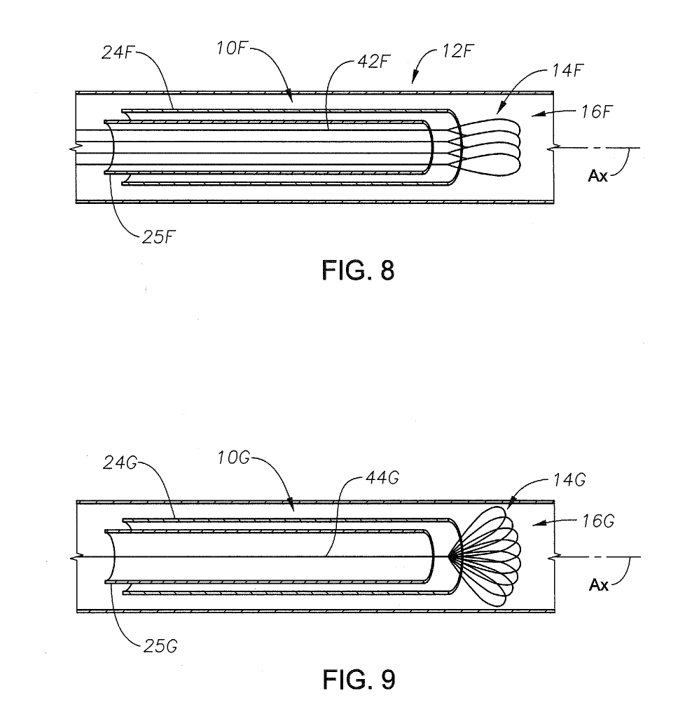

Shown in side partial sectional view in FIG. 8 is an alternate embodiment of snare device 10F where snare element 14F is shown made up of a number of loops 16F and where loops span the inner space of body passage 12F. Moreover, a series of control lines 42F are shown each connecting to a single one of loops 16F so that control of each loop 16F may be performed independently from one another. Control lines 42F pass through outer and inner sheathes 24F, 25F at a distance based axially apart from ends of loops 16F. FIG. 9 shows in a side partial sectional view an example of an alternate embodiment of snare device 100 where snare element 14G includes a plurality of loops 16G, where loops 16G are operated by a single control line 44G that passages through outer and inner sheathes 24G, 25G. Here the loops 16G project radially outward and into contact with wall 200 of passage 14G. Examples exist though where loops 16G are disposed in planes that are parallel with, extend perpendicular to, or are oblique to axis A_{X} of body passage 12G. Further examples exist wherein the loops 16G number about eight, or can range in number from about two to about twenty. Although not shown, a membrane cap 22 (FIG. 1) can be included on the embodiments of the snare devices 10F, 100 of FIGS. 8 and 9. In the loops 16F, 16G of FIGS. 8 and 9, selectively manipulating the associated control lines 42F, 42G can alter a circumference of the loops 16F, 16G. For example, by pulling a one of control lines 42F in a direction away from loops 16F, the circumference of an associated one of loops 16F (i.e. connected to the one of the control lines 42F) can be reduced so that the associated one of the loops 16F can come into close contact with and thus bind an object.

The present invention described herein, therefore, is well adapted to carry out the objects and attain the ends and advantages mentioned, as well as others inherent therein. While a presently preferred embodiment of the invention has been given for purposes of disclosure, numerous changes exist in the details of procedures for accomplishing the desired results. These and other similar modifications will readily suggest themselves to those skilled in the art, and are intended to be encompassed within the spirit of the present invention disclosed herein and the scope of the appended claims.

What is claimed is:

1. A surgical snare device comprising:
a flexible sheath;
a control line in the sheath;
an elongate member coupled with an end of the control line and that is selectively insertable into a body passage; and
loops along the elongate member and defined where the elongate member is curved so that portions of the elongate member are in proximity with one another, and that are disposed at locations distal from the control line and that substantially circumscribe the sheath.

2. The surgical snare device of claim 1, wherein the loops are interconnected to one another.

3. The surgical snare device of claim 1, further comprising a plurality of control lines, wherein a one of the control lines each connect to a one of the loops, and wherein the sheath comprises an outer sheath, and the control lines are disposed within an inner sheath that is within the outer sheath.

4. The surgical snare device of claim 1, further comprising an annular membrane cap that circumscribes the control line, and that is selectively moveable into a deployed configuration that circumscribes a portion of the loops and defines a barrier between the loops and an inner surface of the passage.

5. The surgical snare device of claim 4, wherein the membrane cap comprises a sheet like liner formed into a tubular member and elongate structural elements coupled within the liner, and wherein an open end of the membrane cap has a diameter that is selectively changeable.

6. The surgical snare device of claim 4, wherein the membrane cap is selectively moved from a retracted position within the sheath and to a self-expanding deployed configuration with a portion extending axially outward past the sheath.

7. The surgical snare device of claim 6, wherein a radius of the membrane cap flares outward with distance from the sheath when the membrane cap is in the deployed configuration, and has a trumpet like configuration and with a diameter greater than a diameter of the sheath.

8. The surgical snare device of claim 5, further comprising perforations formed in the liner.

9. The surgical snare device of claim 4, further comprising a control line coupled to the membrane cap for selectively moving the membrane cap into the deployed configuration.

10. The surgical snare device of claim 4, wherein the loops and the membrane cap are disposed within the sheath when being inserted into the body passage.

11. The surgical snare device of claim 1, wherein the loops range in number from about two to about twenty.

12. A surgical snare device comprising:
an elongated tubular sheath selectively insertable into a body passage;
at least one loop deployable in the body passage that are each formed from an elongate filament line, that are controllable by a control line disposed in the sheath, and that occupy spaces across the body passage at a location distal from the sheath; and
an annular membrane cap having at least a portion in the sheath, that circumscribes the control line, and that is selectively deployed from within the sheath to circumscribe the at least one loop.

13. The surgical snare device of claim 12, wherein the annular membrane cap defines a protective layer between the at least one loop and walls of the body passage when in the deployed configuration.

14. The surgical snare device of claim 12, further comprising multiple loops, and wherein the loops are formed from a single elongated element.

15. The surgical snare device of claim 12, further comprising multiple loops, and wherein the loops are of differing diameters.

16. The surgical snare device of claim 12, wherein manipulating the control line changes a diameter of the loop.

17. A method of snaring an object within a body passage comprising:
a. providing a surgical snare device that comprises a series of loops, each of the loops formed by a bend in an elongate member;
b. inserting the surgical snare device into the body passage so that the loops are strategically disposed in spaces that span across the body passage at a location proximate the object; and
c. engaging the object with one of the loops.

18. The method of claim 17, wherein the surgical snare further comprises a membrane cap that comprises a tubular liner, the method further comprising, inserting the membrane cap into the sheath, axially moving the membrane cap with respect to the sheath, thereby causing a diameter of an opening of the membrane cap to expand, further urging the membrane cap in an axial direction so that the membrane cap is between the object and wall of the body passage to define a protective layer for the wall and aid in the capture of the object.

19. The method of claim 17, wherein surgical snare device further comprises loop control lines couple to the elongate members, and a membrane cap control line, the method further comprising controlling the loops by manipulating the loop control lines, and separately controlling the membrane cap deployment and diameter of opening by manipulating the membrane cap control line.

20. The method of claim 17, wherein the loops are disposed in the sheath when the sheath is disposed into the body passage.

21. The method of claim 17, wherein the step of engaging the object with one of the loops comprises manipulating a control line that couples to a one of the loops.

22. The method of claim 17, wherein the step of engaging and capturing the object, the deployed membrane cap encapsulates at least a portion of the loops engaging with the object, the method further comprising, constricting the membrane cap with a control to close an opening so that the captured object is sequestered back within the sheath or held stationary outside the opening of the sheath if the diameter of captured object exceeds the diameter of the sheath.
